(19) United States
(12) Patent Application Publication
(10) Pub. No.: US 2008/0255615 A1
(43) Pub. Date: Oct. 16, 2008

(54) Treatments for Correcting Spinal Deformities

(75) Inventors: Shannon Marlece Vittur, Memphis, TN (US); Randall Noel Allard, Germantown, TN (US); Larry Thomas McBride, Memphis, TN (US)

Correspondence Address: COATS & BENNETT/MEDTRONIC, 1400 CRESCENT GREEN, SUITE 300, CARY, NC, 27518, US

(73) Assignee: Warsaw Orthopedic, Inc., Warsaw, IN (US)

(21) Appl. No.: 11/691,523

(22) Filed: Mar. 27, 2007

(51) Int. Cl. A61B 17/70; A61B 17/58; A61B 5/00; A61B 19/00

(52) U.S. Cl. 606/246; 128/898; 600/300

(57) ABSTRACT

The present application is directed to methods of treating a spinal deformity. The methods may begin by initially testing the spinal deformity. An implant may be chosen to treat the deformity based on the results of the testing. The implant may then be attached to vertebral members to begin treating the deformity. The implant and vertebral members may be monitored afterwards to determine whether the deformity is being corrected, and that the vertebral members remain healthy. If the monitoring warrants, the implant may be adjusted to better correct the deformity and/or prevent damage to the vertebral members.

### BACKGROUND

The present application is directed to treatments for correcting spinal deformities and, more particularly, to treatments that include monitoring implants after insertion within a patient.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various deformities may affect the normal alignment and curvature of the vertebral members. Scoliosis is one example of a deformity of the spine in the coronal plane, in the form of an abnormal curvature. While a normal spine presents essentially a straight line in the coronal plane, a scoliotic spine can present various lateral curvatures in the coronal plane. The types of scoliotic deformities include thoracic, thoracolumbar, lumbar or can constitute a double curve in both the thoracic and lumbar regions. Schuermann's kyphosis is another example of a spinal deformity that affects the normal alignment of the vertebral members.

Implants have been developed to correct the deformities. However, there are no methods for on-going monitoring of the implants after being implanted within the patient. Spinal conditions of the patient may change post-insertion that require the implant to be changed in some manner.

### SUMMARY

The present application is directed to methods of treating a spinal deformity. The methods may begin by initially testing the spinal deformity. An implant may be chosen to treat the deformity based on the results of the testing. The implant may then be attached to vertebral members to begin treating the deformity. The implant and vertebral members may be monitored afterwards to determine whether the deformity is being corrected, and that the vertebral members remain healthy. If the monitoring warrants, the implant may be adjusted to better correct the deformity and/or prevent damage to the vertebral members.

### BRIEF DESCRIPTION OF THE DRAWINGS

FIG. 1 is a schematic diagram illustrating the steps of treating a spinal deformity according to one embodiment.

FIG. 2 is a schematic coronal view of an example of a scoliotic spine.

FIG. 3 is a side view of a stapling system applied along a spinal segment according to one embodiment.

FIG. 4 is a side schematic view of a tethering system applied along a spinal segment according to one embodiment.

FIG. 5 is a schematic view of a deformed spinal segment according to one embodiment.

### DETAILED DESCRIPTION

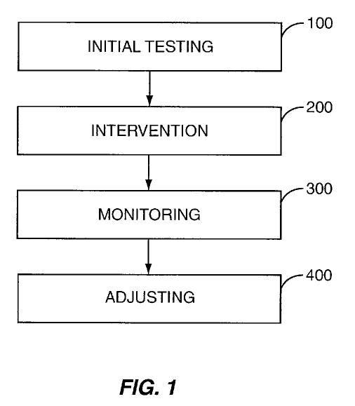

The present application is directed to methods of treating a spinal deformity. FIG. 1 schematically illustrates the steps for treatment that include initial testing of the spinal segment (step 100). This step may include the broad issues of the type of corrective implant, as well as specific issues including placement of the implant and the extent of forces the implant applies to the spinal segment. A second step (step 200) includes attachment of the implant to the spinal segment. This step may also include additional testing to ensure the use of the proper implant and forces or any adjustment to the implant. The second step is generally referred to as intervention because this begins the process of correcting the spinal deformity. A third step (step 300) may include monitoring the implant to determine the extent of correction of the spinal deformity. Monitoring may also include testing the health of the vertebral members and intervertebral discs. A final step (step 400) includes changing one or more aspects of the implant when necessary to ensure that correction is occurring and the vertebral members remain healthy.

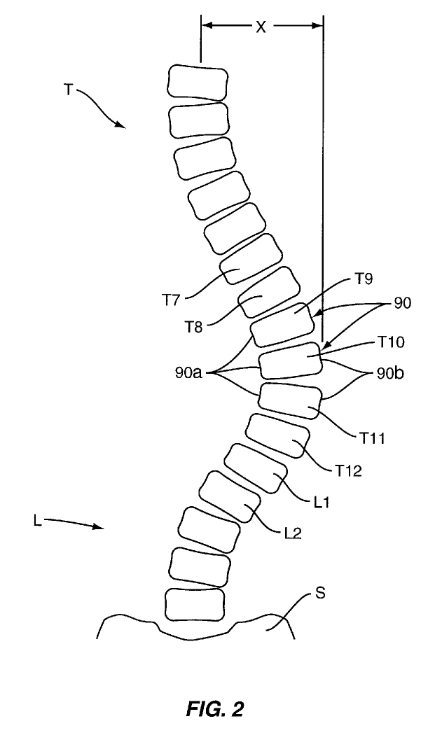

Various types of spinal deformities may be treatable through medical intervention. FIG. 2 illustrates one embodiment of a patient's spine that includes a portion of the thoracic region T, the lumbar region L, and the sacrum S. This spine has a scoliotic curve with an apex of the curve being offset a distance X from its correct alignment in the coronal plane. The spine is deformed laterally so that the axes of the vertebral members 90 are displaced from the sagittal plane passing through a centerline of the patient. In the area of the lateral deformity, each of the vertebral members 90 includes a concave side 90a and a convex side 90b.

Implants to correct spinal curvatures generally apply a force over a length of the spine, referred to as the spinal segment. In one embodiment, the implant is attached to the convex side 90b of the vertebral members 90. These implants apply a compressive force to arrest or at least minimize growth on the convex or “long” side 90b of the spine, thereby allowing the concave or “short” side 90a of the spine to grow and catch up with the long side. In another embodiment, the implant may be attached to the concave side 90a and apply a distractive force to the vertebral members 90 to facilitate growth of the concave or “short” side 90a while not restricting growth on the convex side. Implants may also apply a rotary force to correct axial misalignment of individual vertebral members, or multiple vertebral members along a spinal segment.

The spinal segment receiving treatment may be relatively short and include a single spinal level. In another embodiment, the treated spinal segment is relatively long and extends along multiple spinal levels. In one embodiment, the treatment may include insertion of a single implant, such as at the apex of the deformity. Using FIG. 2 as an example, the implant may span between the T9-T10 vertebral members 90. In another embodiment, multiple implants are attached to the spinal segment. By way of example, a first implant may span between T10-T11, with a second implant spanning T12-L1.

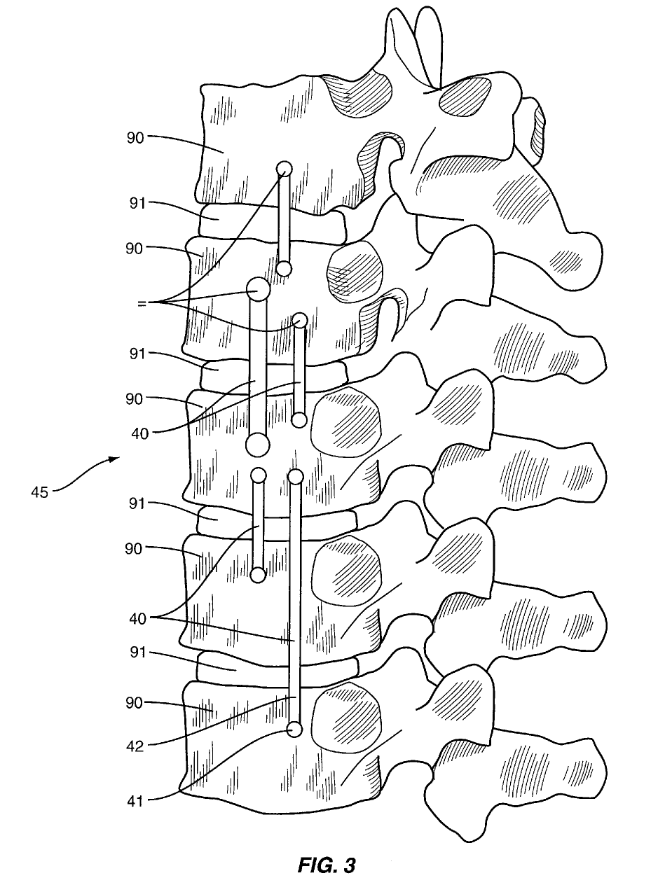

Various implants have been developed to apply the various forces to correct the spinal curvature deformities. The implants may include staples, tethers, rods, plates, and various other members. FIG. 3 illustrates one embodiment of a staple system 45. The systems 45 include one or more staples 40 that each include a pair of arms 41 separated by a base 42. The arms 41 are inserted within the vertebral members 90 with the base 42 spanning across one or more vertebral members 90. The embodiment of FIG. 3 illustrates a system 45 that includes multiple staples 40 that each span across one or more intervertebral discs 91. The bases 42 may be sized to extend across a single or multiple discs 91. In one embodiment, staples 40 may also be positioned intravertebral and remain within a single vertebral member 90. The treated segment includes spinal levels each with one or more staples 40. Various examples of vertebral staples are disclosed in U.S. Pat. Nos. 6,325,805, 6,773,437, and U.S. Patent Application Publication No. 2005/0171539, each of which is herein incorporated by reference.

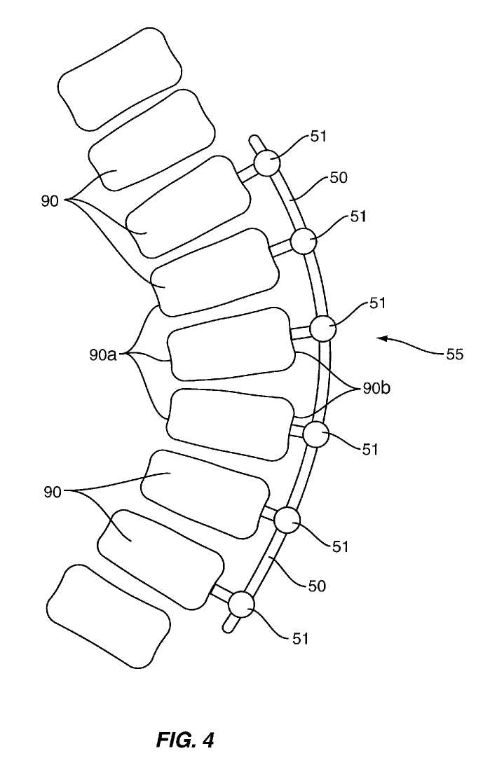

FIG. 4 illustrates an embodiment of a tethering system 55. The tethering system 55 includes an elongated tether 50 sized to extend along two or more spinal levels. Anchors 51 attach the tether 50 to the vertebral members 90. The anchors 51 may be positioned within each or less than each vertebral member 90 along the treated spinal segment. In one embodiment, anchors 51 include a shaft that mounts within the vertebral member 90, and a head that extends outward beyond the vertebral member 90 to receive the tether 50. Each of the anchors 51 may be substantially the same or different in size, shape, and materials. Various types of anchors may be used to attach an implant to the vertebral members 90 such as screws, staples, rivets, pins, and various deployable anchors. The tethering system is an example of a dynamic implant that maintains an intervertebral disc health by allowing for a cyclic motion to pump nutrients in and out of the disc space. Examples of tethering systems include U.S. Pat. Nos. 6,623,484, 6,616,669, and U.S. Patent Application Publication No. 2004/0034351, each of which is incorporated herein by reference.

The various implants are inserted into the patient to correct the spinal deformity. In some successful applications, the spinal deformity is partially or completely corrected. It should also be understood that if the implant fails to correct the spinal deformity but does, in fact, prevent further progression (which includes increase in the magnitude of the curve) it can and should be considered successful.

The first step of the present treatment method is performing initial testing that may include determining the extent of the deformity, the type of implant necessary to correct the deformity, and the amount of force the implant should apply to correct the spinal segment. Initial testing may also assist in distinguishing the choice between two treatment options or interventional technologies. Examples of choices determined through the initial testing may include use a single or multiple staple system, a tether system, flexible rod system, or a distractive device. The initial testing may occur before the surgery, or at the time of surgery but before attachment of an implant.

In one embodiment, initial testing establishes a baseline of the spinal deformity. The baseline may then be used in the future for comparison purposes to determine an amount of change in the spinal deformity, or for comparison to a normal, non-deformed spinal segment.

In one embodiment, the implant includes a release mechanism that releases the amount of force exerted on the vertebral members. The initial testing may determine the appropriate type of release mechanism, or determining the timing and amount of force the implant should exert on the vertebral members before the release mechanism removes or reduces the corrective force. The release mechanism may include a variety of different structures, including a resorbable member that resorbs into the patient after a period of time, or a strength limit that prevent excessive force from being applied to the spine. Examples of implants with release mechanisms include U.S. patent application Ser. No. 11/608,312 filed on Dec. 8, 2006 and entitled “Tethers with Strength Limits for Treating Vertebral Members”, and U.S. patent application Ser. No. 11/676,649 filed on Feb. 20, 2007 and entitled “Resorbable Release Mechanism for Surgical Tether and Methods of Use”, each of which is herein incorporated by reference in their entirety.

The testing techniques may be broadly categorized into measurement testing and biochemical testing. Measurement testing utilizes the physical position of the vertebral members 90 and intervertebral discs 91 to determine the type of implant and necessary corrective forces. The measurement information may be based on various techniques including direct measurement, or radiographic assessment. The measurements are used to calculate various forces being exerted on the spinal segment due to the physical aspects of the spinal curvature. Testing results may determine a stiffness of the spinal curvature along the spinal segment, and the pressure exerted on the spine by the force of the curve.

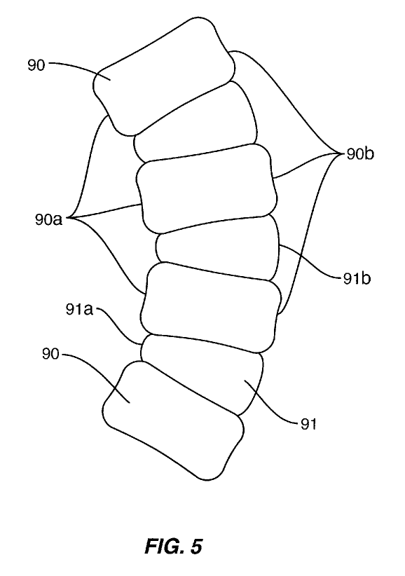

Another type of measurement testing utilizes the displacement of the intervertebral discs 91. FIG. 5 schematically illustrates a section of the spine that includes vertebral members 90 and intervertebral discs 91. Each disc 91 includes a concave side 91a and a convex side 91b due to the curvature of the spine. Measurements are made of each of the sides 91a, 91b and compared with a normal spine to calculate forces applied to the vertebral members 90. The displacement of the disc space between the vertebral members 90 may be obtained with a measurement sensor, such as a differential variable reluctance transducer (DVRT) or a linear variable displacement transducer (LVDT).

The differences in the concave and convex sides of the intervertebral discs 91 may further be tested using magnetic resonance imaging (MRI). MRI techniques utilize a large magnet to polarize hydrogen atoms in the tissues. These techniques are particularly effective for determining water content in the soft tissues of the sides of the intervertebral discs 91. Often times, MRI imaging is superior to other types of radiographic imaging.

Another testing method includes radiograph imaging to compare the spacing of two adjacent vertebral members 90 on the convex and concave sides of the curve. The comparison may determine a desired displacement of one vertebral member relative to the other to achieve the desired correction. Another method includes analyzing the strain across a segment of the spine. In one embodiment, the strain is determined by a DVRT or LVDT measurement sensor.

Biochemical testing is another technique that utilizes the differences between the concave and convex sides of the intervertebral discs 91 and vertebral members 90. One type of biochemical testing includes obtaining tissue samples from each side of the curve for microscopic analysis. The tissue samples may be obtained with a biopsy needle inserted into the vertebral member 90 or intervertebral disc 91. The tissue samples may be obtained at various locations, including the endplate of a vertebral member 90, bone from the vertebral member 90 at a location in proximity to the endplate and growth plate, the annulus of the intervertebral disc 91, and soft tissue in proximity to the vertebral member 90 or intervertebral disc 91. To enhance the testing, a marker injection may be inserted into the patient prior to obtaining the tissue samples. The marker binds to cells factors and may contain radio-isotopes to enhance the testing results. The results of the analysis may be used to determine pressure, force, and distance measurements with comparable results to the measurement testing described above. Results of the biochemical testing may also be used to determine biochemical differences between the convex and concave side of the curve and between the included segments in the deformity and tissue from normal segments.

Various testing may be performed with the tissue samples. Examples include but are not limited to bone morphogenetic proteins (BMP's), collagen (hydroxyproline), proteoglycan content via glycosaminoglycan (GAG), protein content, matrix metallinoproteinases (MMPS), TGF-β1, fibroblast growth factor (FGF), procollagen, parathyroid hormone related protein, pyridinoline and deoxylpyridinoline, and cell density.

Biochemical testing is not limited to tissue samples. Embodiments may also include local fluid draws, serum, and urine analysis.

In some instances, both measurement testing and biochemical testing are utilized during the initial testing. The various testing methods determine the forces acting along the treated spinal segment. In one embodiment, the testing determines the initial correction based on the remaining growth of the vertebral members and desired in vivo correction. Once determined, the proper corrective forces and implant may be applied to the curvature.

The next step (step 200) in the treatment process includes the intervention of introducing the implant into the patient. The intervention includes a surgical procedure to introduce the implant. The procedure may be percutaneous, or may require an open surgical incision.

As part of the surgical intervention, additional testing may be performed. The intervention testing may repeat the initial tests, or may include additional tests not previously performed. In one embodiment, the surgical incision allows for additional, new testing that is otherwise unavailable during non-invasive initial testing. The new testing may provide for more accurate calculations that are otherwise not achievable during the initial testing using measurement and biochemical analysis. The various intervention testing may confirm the selection of the implant, and the parameters for the implant. The testing may also provide an opportunity to adjust the implant intraoperatively.

Generally, in the case of scoliosis, the implants will be positioned on the convex side of the curve. In one embodiment, the implant is implanted with an anterior, minimally invasive (thoracoscopic) procedure on the convex side of the spinal curve. The implant may be delivered into the patient in a minimally invasive approach using thoracoscopic instrumentation. In one embodiment, the implant is delivered with a posterior approach and attached to either the pedicles, lamina, or spinous processes. The tethering system 10 may also be delivered in some combination of both anterior and posterior.

Intervention may also include initializing the implant to apply an initial corrective force to the spinal segment. The initial corrective force may be based on testing (initial and intervention testing). In some embodiments, it has been determined that a range of between about 10 lbs to about 120 lbs is required to correct the deformity. Force levels below this range may not be effective in correcting the spinal deformity. Levels above the range may cause damage to the vertebral members 90 and/or intervertebral discs 91. Various instruments may be used to initialize the implant and apply the corrective force. One example of an instrument is disclosed in U.S. Patent Application Publication 2004/0138666 herein incorporated by reference.

In embodiments with the implant including a release mechanism, intervention may also include setting the release mechanism to prevent damage to the vertebral members 90 or intervertebral discs 91. The release mechanism may be set to prevent an excessive force from being applied, or prevent application of a force that becomes excessive after an extended period of time.

After the surgical procedure is complete, the next step of the method is monitoring the implant (step 300). Monitoring is necessary to ensure the implant does not damage the vertebral members 90 or intervertebral discs 91. Monitoring may also determine whether the implant is correcting the spinal deformity. Monitoring methods include measuring the forces applied to the spinal segment. This may include measurement testing and biochemical testing as explained above.

Monitoring may also include biochemical testing to compare the convex and concave sides of the curve to normal trends for scoliotic spines to determine if the treatment is correcting the deformity. It may also be compared with normal segments of the spine to determine if the samples indicate a return to normal levels. Monitored levels may be compared to baseline levels to determine if the treatment is helping or damaging the segment. These comparisons track the correction of the spinal segment, and may prevent overcorrection of the deformity that could create a deformity in the opposite direction.

The implant is inserted within the patient and applies an initial corrective force to the spinal segment. The force applied by the implant may change over time due to various happenings, such as correction of the spinal deformity and changes to the patient. Certain implants, such as staple and tethering systems 45, 55, are often applied to either infantile or juvenile patients with progressive idiopathic scoliosis. One patient population is prepubescent children (before growth spurt) less than ten years old. Other patient groups upon which the embodiments may be practiced include adolescents from 10-12 years old with continued growth potential. The forces applied by the implants change because the spine with these patients is still growing.

Monitoring may begin immediately after the surgical procedure is completed. This may ensure that the implant applies the proper initial corrective force to the spinal segment. Monitoring may be performed periodically as deemed necessary. This may include weekly, monthly, or even annual assessments of the implant. In one embodiment, regular testing is performed and the change in the applied force of the implant can be closely monitored. When the implant is operating effectively, a gradual increase in the force levels may be observed during each monitoring event. This generally demonstrates correction of the deformity. The regular monitoring schedule also provides for making any necessary changes to the implant in a timely manner, and before the implant may cause damage.

If the monitoring results warrant, the implant is adjusted (step 400). The change may require a revision surgery to properly make the necessary adjustments, or may be performed percutaneously. The adjustments may include a minor increase or decrease in the amount of force being applied by the implant, or may include removal or termination of the implant and its effects. Adjustment may also include changing the vector, or angular moment of the implant.

Minor changes may include tightening or loosening the implant according to the monitoring results. A minor adjustment may occur when the implant is operating effectively, but changes to the patient, deformity, or both require the modification. A major adjustment may occur when damage is being caused by the implant, or the deformity has been corrected. In one embodiment, the adjustment includes cutting the tether 50 or removing a staple 40. In another embodiment, the adjustment includes attaching an additional implant as necessary.

In one embodiment, a release mechanism may be activated to remove or greatly reduce the forces applied by the implant. The activation may be caused by interaction with a physician, or may automatically happen upon the occurrence of a predetermined event. In one embodiment, the implant fails upon application of a force above a preset limit to the spinal segment.

It should be understood that the spinal deformity depicted in FIG. 2 is but one of many types of spinal deformities that can be addressed by the devices and techniques of the present application. Most commonly the devices and methods are expected to be used for either primary thoracic or thoracolumbar curves. They can be used for correction of the thoracic curve as an isolated curve, or the lumbar curve as an isolated curve. The devices may further be used in combination with the shortening of the opposite side of the vertebral member 90.

The devices and methods may be used to treat spinal deformities in the coronal plane, such as a scoliotic spine illustrated in FIG. 2. The devices and methods may also be used to treat deformities in the sagittal plane, such as a kyphotic spine or Scheurmann's kyphosis.

One embodiment includes accessing the spine from an anterior approach. Other applications contemplate other approaches, including posterior, postero-lateral, antero-lateral and lateral approaches to the spine, and accessing various regions of the spine, including the cervical, thoracic, lumbar and/or sacral regions. One embodiment includes a posterior approach to attach an implant to the pedicles, lamina, or spinous process of the vertebral members 90.

The anchors 51 used in the tethering system 55 can be made from a variety of biocompatible and non-resorbable materials. Examples of resorbable materials include polylactide, polyglycolide, tyrosine-derived polycarbonate, polyanhydride, and polyorthoester. Examples of non-resorbable materials include carbon-reinforced polymer composites, shape-memory alloys, titanium, titanium alloys, cobalt chrome alloys, stainless steel, ceramics and combinations thereof.

Various implants may be used to correct the spinal deformity. The implants may include tethers, staples, rods, cables, artificial strands, plates, springs, artificial ligaments, and combinations thereof. The tethers 50 may be rigid, semi-rigid, flexible, partially flexible, resorbable, non-resorbable, superelastic, or include shape-memory material. Tether material may include polymers, such as polyester and polyethylene; superelastic metals, such as nitinol; shape memory alloy, such as nickel titanium; resorbable synthetic materials, such as suture material, metals, such as stainless steel and titanium; synthetic materials, allograft material; and bioelastomer material.

It should be understood that tethering may also be used on older children whose growth spurt is late or who otherwise retain growth potential. It should be further understood that tethering may also find use in preventing or minimizing curve progression in individuals of various ages.

In one embodiment, the methods may include fusionless treatment of the vertebral members to correct the spinal deformity. Another embodiment may include fusion to correct the deformity.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method of treating a spinal deformity comprising:
testing the spinal deformity;
determining an implant to treat the spinal deformity based on testing results;
attaching the implant to vertebral members and applying a corrective force to the spinal deformity;
monitoring the implant after attachment to the vertebral members; and
adjusting the corrective force applied by the implant based on monitoring results.

2. The method of claim 1, wherein the step of testing the spinal deformity includes performing measurement testing on concave and convex sides of the vertebral members.

3. The method of claim 1, wherein the step of testing the spinal deformity includes performing biochemical testing on concave and convex sides of the vertebral members.

4. The method of claim 1, wherein the step of monitoring the implant after attachment to the vertebral members comprises testing an intervertebral disc.

5. The method of claim 1, further comprising obtaining a baseline of the spinal deformity prior to implanting the implant, and the step of monitoring the implant after attachment to the vertebral members comprises comparing the baseline to a monitored status of the vertebral members.

6. The method of claim 1, wherein the step of attaching the implant to the vertebral members comprises attaching one of a staple system and a tether system.

7. The method of claim 1, wherein the step of monitoring the implant after attachment to the vertebral members includes monitoring convex and concave sides of the vertebral members.

8. The method of claim 1, wherein the step of attaching the implant to the vertebral members comprises percutaneously attaching the implant to the vertebral members.

9. The method of claim 1, wherein the step of monitoring the implant includes performing one of measurement testing and biochemical testing.

10. The method of claim 1, wherein the step of adjusting the implant based on the monitoring results includes decreasing a force applied by the implant to the vertebral members.

11. The method of claim 1, wherein the step of adjusting the implant based on the monitoring results includes releasing a force applied by the implant to the vertebral members.

12. The method of claim 1, further comprising monitoring the vertebral members after the implant is attached and adjusting the implant based on a health of the vertebral members.

13. The method of claim 1, wherein the step of attaching the implant to vertebral members and applying the corrective force to the spinal deformity applies a distractive force to a concave side of the vertebral members.

14. The method of claim 1, wherein the step of attaching the implant to vertebral members comprises attaching the implant to a posterior side of the vertebral members.

15. A method of treating a spinal deformity comprising:
determining an implant to treat the spinal deformity based on testing results;
determining a baseline measurement of the spinal deformity;
attaching the implant to a convex side of vertebral members and applying a corrective force to the vertebral members;
monitoring the vertebral members after the implant is attached by comparing monitored results with the baseline measurement; and
adjusting the implant based on the comparison between the monitored results and the baseline measurement.

16. The method of claim 15, further comprising attaching the implant to a posterior side of the vertebral members.

17. The method of claim 15, further comprising testing the convex side of the vertebral members prior to determining the implant.

18. The method of claim 15, further comprising adjusting the implant based on a comparison between the monitored results and testing results from a non-deformed segment.

19. A method of treating a spinal deformity comprising:
testing a spinal segment;
based on the testing, determining an implant to attach to the spinal segment to treat the spinal deformity;
attaching the implant to the spinal segment;
based on the testing, configuring the implant to apply a corrective force to the spinal segment;
monitoring the implant after attachment to the spinal segment; and
adjusting the corrective force applied by the implant based on the monitoring results.

20. The method of claim 19, wherein the step of testing the spinal segment includes performing at least one of measurement and biochemical testing.

21. The method of claim 19, wherein the step of attaching the implant to the spinal segment includes attaching the implant to a convex side of the spinal segment.

22. The method of claim 19, further comprising determining a baseline of the spinal segment prior to attaching the implant to the spinal segment, and comparing the baseline to the monitoring results and determining an amount to adjust the implant.

23. The method of claim 19, wherein the step of attaching the implant to the spinal segment includes attaching a tether system to the spinal segment.

24. The method of claim 19, wherein the step of configuring the implant to apply the corrective force to the spinal segment comprises applying a tension force of between about 10 and 120 pounds to the spinal segment.

25. The method of claim 24, wherein the step of adjusting the corrective force applied by the implant based on the monitoring results includes releasing the implant at a value of between about 10 and 120 pounds.

26. The method of claim 19, wherein the steps of initially testing the spinal segment and monitoring the implant after the attachment to the spinal segment use a common testing technique.

27. The method of claim 19, further comprising monitoring the spinal segment after attachment of the implant and adjusting the corrective force applied by the implant based on a health of the spinal segment.

28. The method of claim 19, further comprising adjusting the corrective force applied by the implant based on a comparison of the monitoring results with testing results from a non-deformed segment.

29. A method of treating a spinal deformity comprising:
testing a spinal segment;
based on the testing, attaching an implant to a convex side of the spinal segment;
based on the testing, configuring the implant to apply a corrective tension force to the spinal segment of between about 10 and 60 pounds;
monitoring the spinal segment after attachment of the implant; and
based on the monitoring results, adjusting the corrective force applied by the implant when the tension force exceeds about 60 pounds.

30. The method of claim 29, further comprising determining the implant to attach to the spinal segment based on the testing.
